(19) United States
(12) Patent Application Publication
(10) Pub. No.: US 2019/0088352 A1
(43) Pub. Date: Mar. 21, 2019

(54) METHOD TO GENERATE NARRATIVE REPORTS FROM EXECUTABLE CLINICAL PATHWAYS

(71) Applicant: KONINKLIJKE PHILIPS N.V., EINDHOVEN (NL)

(72) Inventors: Arvid Randal Nicolaas, Tilburg (NL); Hongchao Nie, Eindhoven (NL)

(21) Appl. No.: 16/085,736

(22) PCT Filed: Mar. 20, 2017
(86) PCT No.: PCT/EP2017/056472

§ 371 Date: Sep. 17, 2018

(30) Foreign Application Priority Data
Mar. 21, 2016 (CN) PCT/CN2016/076871
Jun. 7, 2016 (EP) 16173373.8

(51) Int. Cl. G16H 15/00; G06F 17/28; G16H 10/60

(52) U.S. Cl. CPC G16H 15/00; G16H 10/60; G06F 17/2881

(57) ABSTRACT

A non-transitory storage medium (48) stores a business process (BP) model (46) of a clinical pathway including narrative clinical annotation templates associated to node transitions of the BP model of the clinical pathway. A business process management (BPM) tool (40) comprises a computing device (12) programmed to perform operations including: executing a path for a patient through the BP model of the clinical pathway by traversing nodes of the BP model of the clinical pathway in accord with patient-specific information stored in an electronic medical record (EMR) (10), and generating narrative report content on the clinical pathway for the patient by filling in fields of narrative clinical annotation templates associated to node transitions of the path of the patient through the BP model with patient-specific information stored in the EMR. Narrative report content for parallel branches of the BP model is grouped into a separate paragraph for each branch.

### FIELD

The following relates generally to the electronic medical recording and reporting arts and related arts.

### BACKGROUND

An electronic medical record (EMR) system provides a centralized electronic data repository for storing patient data. Typically, the patient data are indexed by patient, and may be stored in a standard general format such as Health Level Seven International (HL7) and/or in standard domain-specific formats such as DICOM for medical images. Patient data in the EMR may also be stored in less structured formats, such as written medical reports prepared by physicians which may have little or no defined data structure.

Another area in which healthcare is being increasingly automated is the area of clinical diagnosis and monitoring. A clinical decision support (CDS) system may employ a knowledge base in conjunction with expert rules developed by skilled clinicians to provide diagnosis or treatment recommendations for specific patients. In another CDS approach, an electronic clinical pathway is maintained for the patient, and decision support is encapsulated by available flow paths through the clinical pathway. The use of a CDS system can improve diagnosis and treatment, and also serves to enforce compliance with guidelines promulgated by accreditation organizations and medical professional associations and societies.

The following discloses a new and improved systems and methods.

### SUMMARY

In one disclosed aspect, a non-transitory storage medium stores a process model of a clinical pathway including narrative clinical annotation templates associated to node transitions of the BP model of the clinical pathway. A business process management (BPM) tool comprises a computing device programmed to perform operations including: executing a path for a patient through the BP model of the clinical pathway by traversing nodes of the BP model of the clinical pathway in accord with patient-specific information stored in an electronic medical record (EMR), and generating narrative report content on the clinical pathway for the patient by filling in fields of narrative clinical annotation templates associated to node transitions of the path of the patient through the BP model with patient-specific information stored in the EMR. Narrative report content for parallel branches of the BP model is grouped into a separate paragraph for each branch. The model of the clinical pathway may be a business process (BP) stored in Business Process Execution Language (BPEL) and/or in Business Process Model and Notation (BPMN). The BPM tool may be further programmed to run a BP model graphical editor (42) to enable a user to edit the BP model of the clinical pathway and annotate node transitions of the BP model of the clinical pathway with clinical annotation templates associated to the annotated node transitions. Other process model or language can be implemented to serve the purpose for the generation of a narrative medical report with context information included based on the editable process model.

In another disclosed aspect, a non-transitory storage medium stores a BP model of a clinical pathway including narrative clinical annotation templates associated to node transitions of the BP model of the clinical pathway, and instructions executable by a computing device to perform a clinical reporting method in conjunction with an EMR that receives and stores patient-specific information. The clinical reporting method includes: executing a path for a patient through the BP model of the clinical pathway by traversing nodes of the BP model of the clinical pathway in accord with patient-specific information stored in the EMR, generating narrative report content on the clinical pathway for the patient by filling in fields of narrative clinical annotation templates associated to node transitions of the path of the patient through the BP model of the clinical pathway with patient-specific information stored in the EMR, and communicating the generated narrative report content to the EMR. In some embodiments, the non-transitory storage medium stores the BP model of the clinical pathway in at least one of BPEL and a graphical BPMN representation.

In another disclosed aspect, a method is disclosed which operates in conjunction with a BP model of a clinical pathway including narrative clinical annotation templates associated to node transitions of the BP model of the clinical pathway. The method comprises: interfacing with an EMR to record patient-specific information in a data repository of the EMR; on a computer, executing a path for a patient through the BP model of the clinical pathway by traversing nodes of the BP model of the clinical pathway in accord with patient-specific information stored in the EMR; and using the computer, generating narrative report content on the clinical pathway for the patient by filling in fields of narrative clinical annotation templates associated to node transitions of the path of the patient through the BP model of the clinical pathway with patient-specific information retrieved from the EMR. In some embodiments the interfacing includes receiving patient-specific information comprising a medical report via a user interface device, and the method further comprises, during the receiving of the medical report, displaying the generated narrative report content as proposed content for inclusion in the medical report.

One advantage resides in providing automated generation of narrative medical report content for a specific patient.

Another advantage resides in providing an electronic medical record (EMR) with an improved user interface for entering a narrative medical report.

Another advantage resides in facilitating improved reporting on compliance with medical protocols defined by standard clinical pathways.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically shows an electronic medical record (EMR) with a business process management (BPM) tool that executes a path for a patient through a business process (BP) model of a clinical pathway and generates narrative report content.

FIG. 2 diagrammatically shows a portion of an illustrative BP model of a sepsis protocol, with narrative clinical annotation templates associated to some node transitions.

FIG. 3 diagrammatically shows an automated narrative report content generation method suitably performed using the BPM tool of FIG. 1.

### DETAILED DESCRIPTION

As described earlier herein, two existing healthcare automation facets include the increasing use of electronic medical record (EMR) systems, and the increasing use of clinical decision support (CDS) systems. Although EMR and CDS systems provide substantial benefits in patient record-keeping and patient diagnosis/clinical care respectively, they do not address problems relating to medical reporting.

In clinical practice, the preparation of medical reports on a patient is an important facet of health care. Such reports capture and summarize clinical observations drawn by the patient's physician, or by a medical specialist, with respect to the patient's medical condition. A medical report is generally narrative in nature, as narration provides the physician with the greatest level of expressional flexibility and produces a medical report that is readily comprehended by other physicians or other medical personnel.

Attempting to automate, even partially, the generation of medical reports is challenging. The EMR generally contains patient-specific information for preparing a medical report, and indeed the physician is likely to consult the patient's EMR record in preparing a medical report. The EMR may also include reporting user interface capabilities via which the physician can enter (e.g. manually type or dictate via dictation software) a medical report. However, the EMR is not effective in capturing the context of patient information, much less representing the context in narrative form. This context may include factors such as the reason a medical test was ordered, the time at which it was ordered and/or carried out, the patient's medical condition at the time given medical data were generated, any other clinical procedure related information and so forth.

A clinical pathway-based CDS system could in principle provide clinical context. However, the CDS system operates at a coarse level that is not patient-specific. For example, a CDS system for oncology may provide a clinical pathway with nodes depicting various stages or events in an oncology treatment regimen, such as chemotherapy sessions, medical imaging sessions, or so forth. These are relatively high-level events. By contrast, a medical report is usually finer-grained and specific to the patient, for example summarizing vital sign measurements of a patient over the course of a day and presenting clinical conclusions drawn from these measurements, such as by way of illustration a conclusion that the patient is responding well (or not well) to an administered drug. A clinical pathway-based CDS is designed to provide clinical decision support, not retrospective narrative medical reporting. For example, some pathway-based CDS systems display a graphical flow diagram of the salient portion of the clinical pathway, with various nodes annotated with limited patient information.

In approaches disclosed herein, a medical recording and reporting device operates in conjunction with an EMR. The device includes a business process management (BPM) tool that executes a path for a patient through a business process (BP) model of a clinical pathway. The BP model includes narrative clinical annotation templates associated to node transitions of the clinical pathway. To generate narrative report content, fields of narrative clinical annotation templates associated to node transitions of the patient's path through the BP model are filled in with patient-specific information stored in the EMR. The BP model may represent the clinical pathway in detail so that the narrative clinical annotation templates collectively capture detailed clinical context. The purpose of the BP model is to support medical reporting, rather than providing higher level clinical decision support. Accordingly, in some embodiments disclosed herein the user does not directly interface with the BPM tool as it executes the path for the patient—rather, the user interfaces with the EMR, for example via a narrative reporting user interface, and accesses the BPM tool indirectly, via the EMR, to obtain narrative reporting content for possible inclusion in the report or for other purposes.

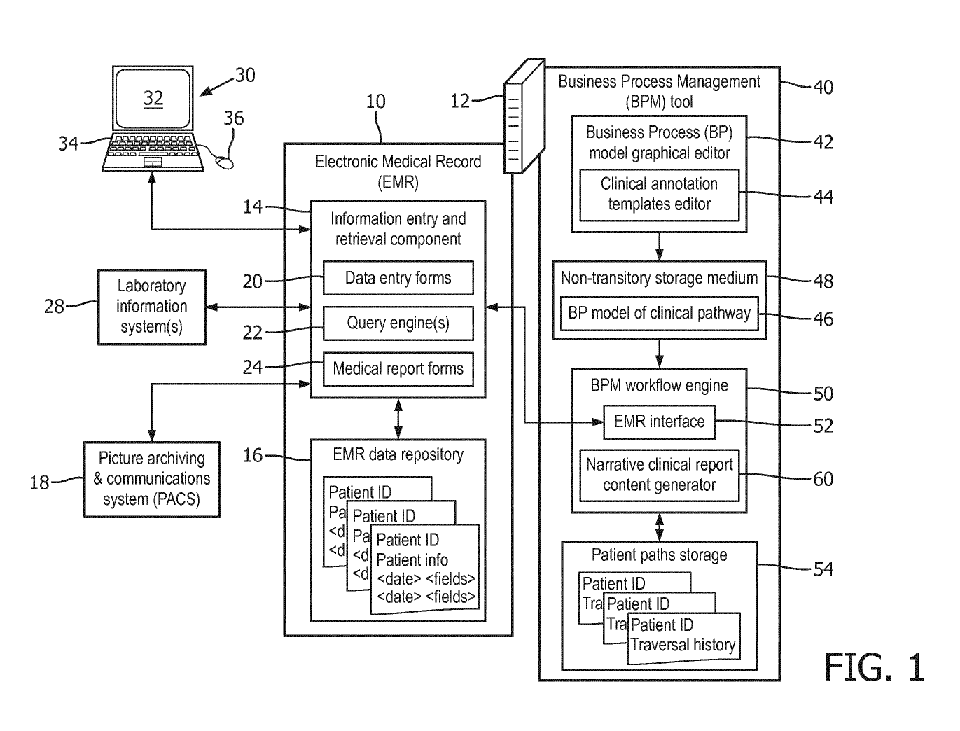

With reference to FIG. 1, an illustrative example of such a system is described. An electronic medical record (EMR) 10 is maintained on a server computer 12. While diagrammatically represented by a single illustrated server computer, the server 12 may more generally be a single server computer or a plurality of interconnected computers, for example a distributed computing system, a cloud computing resource, or so forth. The EMR 10 includes information entry and retrieval components 14 and an EMR data repository 16 in which patient-specific information is stored, indexed by patient, e.g. a patient identification (ID) number or identifier. The patient-specific information includes information such as demographic information about the patient, address information for the patient, and so forth, as well as patient medical data such as vital sign measurements, laboratory test results, medical imaging data (or, in some configurations, links to medical imaging data stored on a separate Picture Archiving and Communication System, PACS 18), narrative medical reports on the patient prepared by physicians (possibly also including narrative report content automatically generated as disclosed herein), or so forth. The stored patient-specific information may include, e.g., medical data, clinical reports, links to medical data, and links to clinical reports, and/or so forth. The patient-specific data are typically, although not necessarily, time stamped (for example, demographic information such as gender or ethnicity may not be amenable to time stamping), and where feasible the patient-specific information is preferably stored in a structured format, for example with data-typed HL7 fields storing particular types of data. The information entry and retrieval components 14 include a range of components (i.e. tools) providing various ways to enter patient data into the EMR 10 and various ways to retrieve and present patient data contained in the EMR. The illustrative information entry and retrieval components 14 include diagrammatically indicated data entry forms 20 (e.g., web-based forms with data-typed data entry fields facilitating structured data input), query engines 22 (e.g., structured query language, SQL, query engines if the data repository 16 is a relational data management system, RDBMS), and clinical reporting forms 24 (e.g., a word processing interface, and/or a more structured reporting environment with freeform text entry fields). These are merely illustrative interfacing tools and additional or other such components or tools are contemplated. The information entry and retrieval components 14 interface with various external systems or components to perform such interfacing, for example interfacing with the aforementioned PACS 18 to retrieve medical images and related data, and/or with one or more laboratory information systems 28 such as those supporting hematology, histopathology, cytopathology, microscopy, or other domain-specific laboratories. Again, these are merely illustrative examples 18, 28 and numerous other facilities both internal and external to the hospital may be similarly interfaced by the information entry and retrieval components 14. Furthermore, the information entry and retrieval components 14 typically interface with computer workstations or other data entry terminals, such as a representative EMR computer workstation 30 including a display 32 and one or more user input devices such as an illustrative keyboard 34 and a mouse 36 or other pointing device. A physician, nurse, hospital clerical staff, or so forth may use the workstation 30, for example, to fill out one of the electronic data entry forms 20 or to enter a medical report via one of the medical report forms 24, and/or to retrieve stored patient information by entering a suitable query via one of the query engines 22.

As used herein, the term “electronic medical record” or EMR is to be understood as encompassing integrated medical information systems providing patient data aggregation and storage, whether such system is referred to as an electronic medical record, or an electronic health record (EHR), or by some other nomenclature.

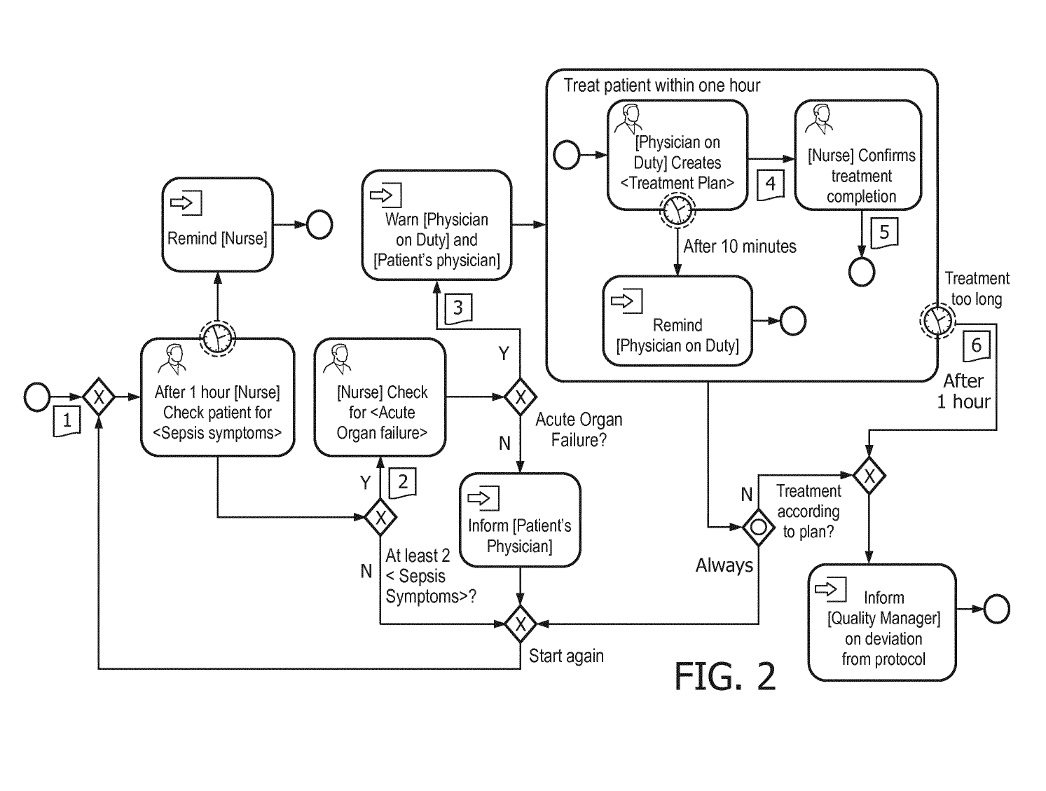

As already discussed, the EMR 10 provides patient data storage and retrieval, but does not provide for automated generation of narrative medical report content in a natural language (e.g. English, Chinese, French, or so forth) including relevant contextual information and in some instances clinical conclusions that may be reasonably drawn. Rather, a Business Process Management (BPM) tool 40 is provided to generate narrative report content. The BPM tool 40 may execute on the same computer server 12 that hosts the EMR 10, as shown, or the BPM tool may reside on a different computer that is communicatively connected with the EMR computer(s). The BPM tool 40 may be implemented using substantially any BPM development suite, such as Bonita BPM (available from Bonitasoft, Inc.), the Oracle Business Process Management Suite (available from Oracle Corp.), or so forth. The BPM suite provides or is operatively connected with a Business Process (BP) model graphical editor 42 which provides for constructing or editing a BP model, for example in a graphical Business Process Model and Notation (BPMN) representation. BPMN represents the BP model using a flow chart format including flow objects representing events, activities, gateways (e.g. decision nodes), or so forth; and connector or connecting objects representing process and/or data flow between the flow objects (see, e.g., FIG. 2 described in detail later herein). More generally, the term “node” is used herein to generically denote a process event, task, or other point in the BP model (e.g. a flow object in BPMN graphical notation); while the term “node transition” is used herein to denote process flow into or out of a node, typically in accord with a connector object that connects with the node in BPMN graphical notation.

The BP model graphical editor 42 is modified as disclosed herein to incorporate a narrative clinical annotation templates editor 44 that allows for adding narrative clinical annotation templates associated to node transitions of the BP model. A physician or other medical professional suitably uses the BP model graphical editor 42 to construct and/or edit a BP model of a clinical pathway with narrative clinical annotation templates associated to chosen node transitions using the templates editor 44. In addition to the narrative clinical annotation templates editor 44, the graphical editor 42 may include other extensions, plug-ins, or the like (not shown) providing flow object definitions for nodes (e.g. flow objects) implementing communication with the EMR 10—such EMR access definitions may for example be created by configuring existing BPMN data objects to interface the EMR 10 with which the BP model will interact.

The resulting constructed or edited BP model 46 of the clinical pathway with narrative clinical annotation templates is stored in a non-transitory storage medium 48 of, or accessible by, the BPM tool 40. The storage medium 48 may, for example, comprise a hard disk, RAID, or other magnetic storage medium, an optical disk or other optical storage medium, a flash memory or other electronic storage medium, various combinations thereof, or so forth. The BP model 46 may be stored on the non-transitory storage medium 48 in BPMN and/or in a compiled executable language such as Business Process Execution Language (BPEL). A BPM workflow engine 50 reads and executes the stored BP model 46 of the clinical pathway with narrative clinical annotation templates. This is done for a particular patient—thus, multiple “instances” of the executing BP model 46 may be extant at any given time. In executing the BP model 46 for a particular patient, an EMR interface 52 implements interfacing with the EMR 10 to perform patient data retrieval or write operations such as retrieving patient data for the particular patient required at a particular node.

As the BP workflow engine 50 executes the BP model 46 for a particular patient, various data are collected for various traversed nodes (e.g. flow objects in BPMN), and/or data may be generated at various traversed nodes. The actual path traversed for a particular patient through the BP model 46 of the clinical pathway is typically also stored. This may also be referred to as the patient's traversal history. This patient-specific information is stored in a patient paths storage 54, e.g. indexed by patient ID. Like the non-transitory storage medium 48, the patient paths storage 54 is suitably a non-transitory storage medium such as, for example, a hard disk, RAID, or other magnetic storage medium, an optical disk or other optical storage medium, a flash memory or other electronic storage medium, various combinations thereof, or so forth.

The computational components of the BPM tool 40, e.g. the BP model graphical editor 42 and the BPM workflow engine 50 and their constituent components, are also suitably stored on a non-transitory storage medium (not shown) as executable code (i.e. a program) readable and executable by the computer 12. Again, the non-transitory storage medium may, for example, comprise a hard disk, RAID, or other magnetic storage medium, an optical disk or other optical storage medium, a flash memory or other electronic storage medium, various combinations thereof, or so forth. It should be noted that in some embodiments the BP model 46 is generated at a separate model editor (for example, provided by a commercial vendor who markets the EMR 10 with integrated BPM tool 40), in which case the BPM tool 40 that is in operative communication with the EMR 10 may optionally omit the BP model graphical editor 42. Even in the case of offline BP model generation, however, the BP model graphical editor 42 may optionally still be provided to enable subsequent editing or updating of the BP model 46 and/or its narrative clinical annotation templates.

Execution of the BP model 46 for a particular patient provides a mechanism to track progression of the patient through the modeled clinical pathway. The BP model 46 can be constructed at a finely detailed level so as to capture relatively “mundane” events such as several times-daily administration of various medications, various vital sign measurements performed at various times over the day, and so forth; as well as capturing higher-level detail such as when the patient undergoes an MRI examination, or a radiation therapy treatment session or so forth. Narrative clinical annotation templates are also associated to some node transitions of the path of the patient through the BP model 46 of the clinical pathway. These narrative clinical annotation templates are created and associated to node transitions using the clinical annotation templates editor 44 during creation or editing of the BP model 46 of the clinical pathway. These narrative clinical annotation templates are used to automatically generate narrative report content on the clinical pathway for the patient. This is done by filling in fields of narrative clinical annotation templates associated to node transitions of the path of the patient through the BP model 46 of the clinical pathway with patient-specific information stored in the EMR 10.

The narrative report content can be generated in (approximately) real-time, that is, as the BP model 46 is executed for the patient, each time a node transition having an associated narrative clinical annotation template is traversed during the execution, a narrative clinical report content generator component 60 of the BP workflow engine 50 (as shown, or alternatively the narrative clinical report content generator 50 may be separate from the workflow engine) fills in the fields of the narrative clinical annotation template associated to the currently executing node transition and stores the narrative report content generated by filling in the template in the patient's record in the patient paths storage 54. In this case, when a physician is preparing a medical report for the patient, e.g. using the illustrative EMR computer workstation 30 and the one of the medical report forms 24, the user can select an option to retrieve narrative clinical report content from the EMR. In response to this selection, the EMR/BPM tool interfacing components 14, 52 are used to retrieve the narrative report content stored in the patient's record in the patient paths storage 54.

In a variant embodiment, the narrative report content is generated retrospectively. In this case, the templates are not filled in at the time the associated node transition is executed. Rather, when the physician selects the option to retrieve narrative clinical report content from the EMR, the the narrative report content is generated at the time of this selection (i.e. retrospectively) by re-tracing the previously executed path for the patient through the BP model of 46 the clinical pathway. This is possible because the traversal history is stored in the patient paths storage 54. As the path for the patient through the BP model 46 is re-traced, fields of narrative clinical annotation templates associated to node transitions of the re-traced path are filled in with patient-specific information stored in the EMR 10 in order to generate the narrative report content.

In either the real-time or retrospective embodiments, the generated narrative report content is communicated to the EMR 10 via the interfacing components 14, 52, and is displayed to the physician on the display 32. In one approach, suitable for use in the described context in which the physician is drafting a medical report for the patient, the generated narrative report content is proposed to the physician by displaying the generated narrative report content on the display 32, and the proposed narrative content is copied into the medical report under draft (or otherwise stored in the EMR data repository 16) in response to an assent to the proposal received from the physician via the user interface device(s) 34, 36. In such embodiments, the physician or other user does not directly interface with the BPM tool 40 and the BPM tool 40 is not programmed to operate the user interfacing device 30 to display the generated narrative report content; rather, the BPM tool 40 is programmed to communicate the generated narrative report content to the EMR 10 and the EMR handles the user interfacing to present the generated narrative report content to the physician or other user. (In other, alternative embodiments, it is contemplated for the physician to directly interface with the BPM tool).

A further matter that can arise in generating narrative report content as disclosed herein is how to address parallel paths (branches) of the BP model 46. Parallel paths are common in clinical pathways—for example, upon diagnosing the patient with a particular type of cancer, the physician may order: a biopsy providing tissue samples for histopathology testing; medical imaging to assess the presence/characteristics of tumor(s); and initial chemotherapy or some other initial therapy (which will likely be modified upon receipt of the laboratory test and imaging data). Thus, the decision node representing the cancer diagnosis has several outgoing node transitions to parallel branches: one branch modeling the biopsy/histopathology; one branch modeling the medical imaging; and one branch modeling the initial treatment.

In illustrative examples herein, such parallel paths or branches are handled by the narrative clinical report content generator 50 as follows. Where the path for the patient through the BP model 46 of the clinical pathway includes two or more parallel branches of the BP model representing actions of the clinical pathway taken in parallel, the narrative report content generated by filling in fields of narrative clinical annotation templates associated to node transitions of each branch is grouped into a paragraph for that branch. This generates a separate narrative report content paragraph for each branch of the two or more parallel branches. This approach recognizes that each parallel branch typically represents a relatively self-contained sub-process, often performed by a designated set of actors. Thus, grouping the narrative report content of each branch into its own paragraph is likely to result in a semantically connected narrative describing the sub-process represented by the branch.

In the following, some further illustrative examples are described. In general, the system includes: the EMR 10 containing the clinical patient data, like age or other demographic data, laboratory test results, information on diagnostic and/or therapeutic procedures performed on the patient, or so forth; the BP model 46 based on the clinical pathway that describes the steps to be taken to treat patients for a certain condition; the BP model editor 42 which interfaces the BP model author to add specific annotations to parts (e.g. node transitions) of the BP model 46 (e.g. via the templates editor 44 in the illustrative example); the BP workflow engine 50 which executes the BP model 46 for a particular patient, and is integrated with the EMR 10; and the report generator module 60 configured to read data from the workflow engine 50, possibly extended with additional data from the EMR 10.

The BP model 46 of the clinical pathway is extended with process annotations (represented by narrative clinical annotation templates) that allow the BP model author to include sentence parts or other narrative content that can be used for report generation. Fields of the narrative clinical annotation templates are filled in during execution (or re-tracing) to incorporate, into the narration, patient-specific information from the EMR 10 or other sources, such as vital sign readings, date information, laboratory test data, or so forth. Typically, the most important pieces of information for inclusion in a medical report pertain to outgoing transitions of nodes, as they represent a completed action or decision that is to be summarized in a medical report. Incoming node transitions may also be of importance as they represent the data leading to the task, event, or the like represented by the node (moreover, in many cases an outgoing node transition of one node corresponds to the incoming node transition of a next node). Further, it is recognized herein that information at decision nodes is typically of particular importance for inclusion in the medical report, as decision nodes are where the succeeding course of actions is determined.

By way of illustration, consider a task that is to be performed by a nurse, such as “Check blood pressure”. In this case, the task is represented by a node (e.g. a flow object in BPMN), and the narrative clinical annotation template on the outgoing node transition is suitably “The nurse measured a blood pressure reading of <BP> at <time>”, where <BP> and <time> are fields of the annotation template that are filled in by the actual blood pressure reading and the actual time, respectively. The data to be filled in are suitably retrieved from the EMR data repository 16 via the interfacing components 14, 52.

A narrative clinical annotation template may be conditional, so that it generates narrative report content only if some condition specified in the template is met. For example, in the previous blood pressure measurement example, it may be unnecessary to include the blood pressure in a medical report if the blood pressure reading is in the normal range for the patient. Hence, for a BP model node which is a decision point that decides “Patient's blood pressure too high?”, the outgoing node transition annotation template may be constructed to generate: (1) narrative report content of the form “Blood pressure reading <BP> is high” if the blood pressure reading is greater than some threshold, or (2) no narrative report content if the blood pressure reading does not exceed the threshold.

When requested, the narrative clinical report content generator 60 generates a narrative report of processes executed or processes in execution. In the retrospective embodiment, it does so in the following way. Starting at a start node of the BP model 46, the executed path through the BP model 46 is followed (i.e. re-traced). For each narrative clinical annotation template that is encountered, the fields of the annotation template are filled in to generate narrative report content, preferably in the form of one or more sentences defined by the template. (As just noted, if the template is conditional then narrative report content is generated only if the condition is met, e.g. blood pressure above threshold). The narrative report content generated at each such node transition is concatenated together to generate an ongoing narration. If a traversed node has a plurality of outgoing node transitions (i.e. parallel branches), then this process is performed for each branch to generate a separate paragraph for that branch, and the paragraphs are combined to form multi-paragraph narration of the parallel branches grouped by branch. This generally provides more coherent narration as compared with bits of information from each branch being mixed together. The generated narrative report content is presented to the physician or other end user, in the illustrative embodiment by forwarding it to the EMR 10 which proposes the generated narrative content for inclusion in a medical report under-draft, and/or for inclusion in the patient record in the EMR data repository 16.

With reference now to FIG. 2, an example is presented employing a BP model for a clinical pathway comprising a sepsis protocol. FIG. 2 illustrates a portion of the BP model, shown using BPMN notation such as might be seen by the model developer interacting with the BP model graphical editor 42. There are a number of Human Tasks represented in the graphical representation of FIG. 2 by rectangles with a “person” icon shown in the upper left-left. A decision node is represented by an “X” circumscribed by a diamond. A timer is indicated by a “clock” icon shown at an edge of a node, and indicates that a node transition is triggered by the elapsing of the indicated time interval. Process (or sub-process) start and stop nodes are indicated by small circle nodes.

With continuing reference to FIG. 2, narrative clinical annotation templates associated to node transitions using the clinical annotation templates editor 44 are indicated by “document” icons, where the “document” icon is a sheet of paper cut-off at the bottom by a curved line. For ease of description herein, narrative clinical annotation template icon is enumerated—there are six such narrative clinical annotation templates in FIG. 2 enumerated 1 . . . 6. (Such enumeration is optional in the actual BP model BPMN representation, as the user can select to view/edit a narrative clinical annotation template by clicking on its icon using a mouse pointer or by some other user selection operation.) The six illustrative narrative clinical annotation templates are described in turn below.

The narrative clinical annotation template enumerated as “1” is associated to a node transition from the process entry node at the far left in FIG. 2. The narrative clinical annotation template may suitably read “Sepsis protocol report on patient <patient_name>, age <patient_age>: \CR\CR The patient entered the sepsis protocol in <location> on <time> diagnosed with <primary_diagnosis>.” The fields <patient_name> and <patient_age> are filled in with the patient name and age information from the patient's EMR record. The notation \CR indicates a carriage return. The <location> and <time> fields are filled in with the patient's location at diagnosis and the time of diagnosis, respectively, and the <primary_diagnosis> field is filled in with the primary diagnosis.

The narrative clinical annotation template enumerated as “2” is associated to a node transition from the decision node labeled “At least 2<Sepsis symptoms>”. A suitable narrative clinical annotation template here may be “On <time>, the nurse vital sign check revealed that the patient was at risk of severe sepsis, based on the following criteria: <list of abnormal values>.” The field <list of abnormal values> may be filled in by searching patient data acquired in a narrow time window around <time> to identify the abnormal values.

The narrative clinical annotation template enumerated as “3” is associated to a node transition from the decision node labeled “Acute organ failure?” A suitable narrative clinical annotation template may be “The result of the ordered blood test confirmed the presence of Acute Organ Failure, based on the following criteria: <list of abnormal values>. <Patients_physician> was informed at <detection_time>.”

The narrative clinical annotation template enumerated as “4” is associated to a node transition from the flow object labeled “[Physician on duty] created <Treatment Plan>”. A suitable narrative clinical annotation template may be “<Patients_physician> created the following treatment plan at <time>: <list of medications>.” The field <list of medications> may be filled in from a prescription entry logged in the EMR 10 at about the time of execution of the flow object “[Physician on duty] Creates <Treatment Plan>”.

The narrative clinical annotation template enumerated as “5” is associated to a node transition from the flow object labeled “[Nurse] Confirms treatment completion”. A suitable narrative clinical annotation template may be “The nurse administered the medication at <administration time>.” In this case <administration time> is filled in with the time stamp of the medication administration entry in the patients' EMR record.

The narrative clinical annotation template enumerated as “6” is associated to a node transition from the timer-governed node transition from the flow object labeled “Treat patient within one hour.” A suitable narrative clinical annotation template may be “The time between the detection of severe sepsis and the administration of antibiotics was longer than the target of the clinical pathway.”

As each node transition associated to the narrative clinical annotation templates enumerated 1 . . . 6 is traversed, corresponding narrative report content is generated. In this way, the following aggregate narrative report content may be generated:
Sepsis protocol report on patient John Doe, age 48:
The patient entered the sepsis protocol in the General Ward on Sunday, March 26 at 6:24 PM diagnosed with cardiac surgery. On Monday, March 27 at 10:36 AM, the nurse vital sign check revealed that the patient was at risk of severe sepsis, based on the following criteria:
   Temperature of 39.9 C (above the limit of 36.6 C)
   Respiration rate of 35 rpm (above the limit of 30)
The result of the ordered blood test confirmed the presence of Acute Organ Failure, based on the following criteria:
   Leucocytes: 36.540 (above the limit of 35.000)
Dr. Smith was informed at 10.45 AM. Dr. Smith created the following treatment plan at 10.56 AM:
   Antibiotic: Xanatex 35 mg
The nurse administered the medication at 11:55 AM. The time between the detection of severe sepsis and the administration of antibiotics was longer than the target of the clinical pathway.

Although not shown in the diagrammatically indicated narrative clinical annotation templates enumerated 1 . . . 6 above, the fields of these templates may optionally contain formatting delimiters or the like, e.g. specifying the choice of time output (HH:MM, or HH:MM:SS, et cetera), formatting for patient data, or so forth. Templates may contain other features such as regular expressions, e.g. for the template enumerated 6, it is contemplated to add a difference expression to compute and narrate quantitatively how much the time interval between detection of severe sepsis and administration of antibiotics was compared with the target of the sepsis clinical pathway.

As another example, the narrative clinical annotation template may narrate a checklist, in which several tasks are to be completed before next steps are taken. In such a case, the narrative report content can include the tasks that were performed, with fields to be filled in identifying the actor who performed each task, and at which times, and optionally also, if a task was not performed, a reason why. An example of such a check list is:
At <8.26 PM>, the <physician W.> created a <treatment plan> after the following tasks were completed:
   Measure Blood Pressure, performed by <nurse P.> at <8.13 PM>.
   Retrieve Lab Test result, performed by <nurse T.> at <8.16 PM>.
   Not completed: Provide ACE Inhibitor medication. Dismissed by <physician W. > with reason <patient has allergy>.
      where the angle brackets (< . . . >) again denote fields.

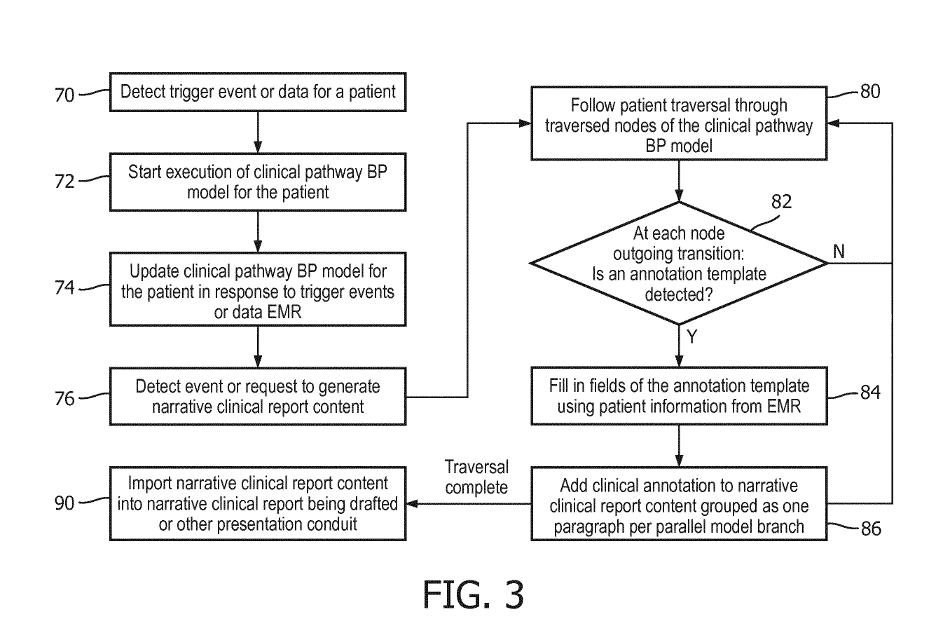

With reference to FIG. 3, an automated narrative report content generation method suitably performed by the system of FIG. 1 is described. In an operation 70 a trigger event, or generation of trigger data, is detected which triggers operation 72 in which execution of the BP model 46 of the clinical pathway is started for the patient. In the sepsis protocol example above, the trigger event is detection of vital signs indicative of risk of severe sepsis, and the operation 72 starts execution of the BP model of the sepsis protocol for the patient. This also entails creating a patient path record for the patient in the patient paths storage 54 in order to record the patient's path through the sepsis protocol. In an operation 74 the BP model 46 is executed by the workflow engine 50. This entails detecting trigger events or data causing transitions from one node to another, and recording the traversal history in the storage 54. In an operation 76, an event or request is detected that triggers the generation of narrative report content. (This is employing the retrospective approach in which the path is re-traced). The triggering event or request may, for example, be a physician opening one of the medical report forms 24 via the user interface 30 which is designed to record subject matter of the clinical pathway being modeled by the BP model 46. Alternatively, the triggering event or request may be an affirmative action by the physician such as selecting an option on the user interface for the EMR to propose narrative report content. (Again, in the illustrative embodiment the physician interacts with the EMR 10, and the BP tool 40 is hidden so that the physician perceives the EMR as generating the narrative report content).

In an operation 80, the patient traversal history is retrieved from the patient paths storage 54 and the patient traversal is re-traced starting at the entry node of the BP model 46. For each re-traced node, at a decision 82 the outgoing transition (e.g. the outgoing BPMN connecting object) is examined to determine whether it has an associated narrative clinical annotation template. If not, then process flows back to the operation 80 to re-trace to the next node along the patient's traversal path. On the other hand, if the decision 82 detects a narrative clinical annotation template associated with the outgoing node transition then in an operation 84 the fields (if any) of the narrative clinical annotation template are filled in with patient-specific information from the EMR 10, and in an operation 86 the resulting narrative report content is added to the (cumulative) narrative report content.

A given node may have two or more outgoing node transitions—if so then each outgoing node transition is examined in turn in the decision 82 and processed as per operations 84, 86 if there is an associated narrative clinical annotation template. If this results in identifying parallel branches, then each branch is processed separately using the (looping) operations 80, 82, 84, 86 to generate a separate narrative report content paragraph for each branch, and the paragraphs are collected to generate the cumulative narrative report content.

When the patient's traversal history to the present time has been fully re-traced, then in an operation 90 the resulting cumulative narrative report content is imported to the medical report under draft in the EMR 10, or is otherwise conveyed to the EMR 10 and used (e.g., stored in the EMR repository 16).

The method of FIG. 3 employs the retrospective re-tracing approach for generating the narrative report content. If instead the real-time approach is used, then the operations 80, 82, 84, 86 are integrated in real-time into the BP model execution 74, and in response to the request 76 the narrative report content already generated and stored in the patient paths storage 54 is read out and imported to the EMR 10.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A medical recording and reporting device comprising:
a storage medium storing a process model of a clinical pathway including narrative clinical annotation templates associated to node transitions of the process model of the clinical pathway; and
a process management tool executable on a computing device and having:
a workflow engine for executing a path for a patient through the process model of the clinical pathway by traversing nodes of the process model of the clinical pathway in accord with patient-specific information stored in an electronic medical record (EMR), and
a content generator for generating narrative report content on the clinical pathway for the patient by filling in fields of narrative clinical annotation templates associated to node transitions of the path of the patient through the process model of the clinical pathway with patient-specific information stored in the EMR.

2. The medical recording and reporting device of claim 1 further comprising:
said electronic medical record (EMR) comprising a computing device programmed to receive patient-specific information comprising one or more of medical data, clinical reports, links to medical data, and links to clinical reports and to store the received patient-specific information in an EMR data repository.

3. The medical recording and reporting device of claim 2 wherein the EMR is further programmed to operate a user interface device to display the generated narrative report content.

4. The medical recording and reporting device of claim 2 wherein the EMR is further programmed to:
propose the generated narrative report content by operations including displaying the generated narrative report content on a display; and
store the generated narrative report content in the EMR data repository in response to an assent to the proposal received via the user interface device.

5. The medical recording and reporting device of claim 1 wherein the process management tool is programmed to perform a further operation of communicating the generated narrative report content to the EMR, and the process management tool is not programmed to operate a user interfacing device to display the generated narrative report content.

6. The medical recording and reporting device of claim 1 wherein the process management tool is programmed to perform further operations including:
storing the path for the patient through the process model of the clinical pathway in a patient paths storage;
wherein the narrative report content is generated by re-tracing the stored path for the patient through the process model of the clinical pathway and filling in fields of narrative clinical annotation templates associated to node transitions of the re-traced path of the patient through the process model of the clinical pathway with patient-specific information stored in the EMR.

7. The medical recording and reporting device of claim 1 wherein the process management tool fills in fields of the narrative clinical annotation templates as the associated node transitions are traversed to generate the narrative report content during execution of the path for the patient through the process model of the clinical pathway, and the process management tool is programmed to perform further operations including:
storing the narrative report content generated during execution of the path for the patient through the process model of the clinical pathway in a patient paths storage.

8. The medical recording and reporting device of claim 1 wherein the generating of narrative report content on the clinical pathway for the patient further includes:
where the path for the patient through the process model of the clinical pathway includes two or more parallel branches of the process model representing actions of the clinical pathway taken in parallel, grouping narrative report content generated by filling in fields of narrative clinical annotation templates associated to node transitions of each branch into a paragraph for that branch to generate a separate narrative report content paragraph for each branch of the two or more parallel branches.

9. The medical recording and reporting device of claim 1 wherein the non-transitory storage medium stores the process model of the clinical pathway in Business Process Execution Language (BPEL).

10. The medical recording and reporting device of claim 1 wherein the PM tool is programmed to perform further operations including:
running a process model graphical editor to enable a user to:
edit the process model of the clinical pathway, and
annotate node transitions of the process model of the clinical pathway with clinical annotation templates associated to the annotated node transitions.

11. The medical recording and reporting device of claim 10 wherein the BP model graphical editor is configured to edit the process model of the clinical pathway represented in a graphical Business Process Model and Notation (BPMN) representation.

12. A non-transitory storage medium storing:
a process model of a clinical pathway including narrative clinical annotation templates associated to node transitions of the process model of the clinical pathway; and
instructions executable by a computing device to perform a clinical reporting method in conjunction with an electronic medical record (EMR) that receives and stores patient-specific information, the clinical reporting method including:
executing a path for a patient through the BP model of the clinical pathway by traversing nodes of the BP model of the clinical pathway in accord with patient-specific information stored in the EMR,
generating narrative report content on the clinical pathway for the patient by filling in fields of narrative clinical annotation templates associated to node transitions of the path of the patient through the process model of the clinical pathway with patient-specific information stored in the EMR, and
communicating the generated narrative report content to the EMR.

13. The non-transitory storage medium of claim 12 wherein the non-transitory storage medium stores the process model of the clinical pathway in at least one of Business Process Execution Language (BPEL) and a graphical Business Process Model and Notation (BPMN) representation.

14. A method operating in conjunction with a process model of a clinical pathway including narrative clinical annotation templates associated to node transitions of the process model of the clinical pathway, the method comprising:
interfacing with an electronic medical record (EMR) to record patient-specific information in a data repository of the EMR;
executing a path for a patient through the process model of the clinical pathway by traversing nodes of the process model of the clinical pathway in accord with patient-specific information stored in the EMR; and
generating narrative report content on the clinical pathway for the patient by filling in fields of narrative clinical annotation templates associated to node transitions of the path of the patient through the process model of the clinical pathway with patient-specific information retrieved from the EMR.

15. The method of claim 14 wherein the interfacing includes receiving patient-specific information comprising a medical report via a user interface device, and the method further comprises:
during the receiving of the medical report, displaying the generated narrative report content as proposed content for inclusion in the medical report.
